SEQUENCE LISTING (1 sequence)

SEQ ID NO: 1 - protein, 8 aa, Antigenic peptide Ovalalbumin
SIINFEKL

(19) United States
(12) Patent Application Publication
(10) Pub. No.: US 2002/0106376 A1
(43) Pub. Date: Aug. 8, 2002

(54) Therapeutic or prophylactic composition of fibronectin-binding protein 1 (SFBI) as adjuvant

(75) Inventors: Eva Medina, Braunschweig (DE); Gursharan S. Chhatwal, Braunschweig (DE); Carlos A. Guzman, Braunschweig (DE)

Correspondence Address: MARSHALL, GERSTEIN & BORUN, 6300 SEARS TOWER, 233 SOUTH WACKER, CHICAGO, IL, 60606-6357, US

(73) Assignee: Gesellschaft fuer Biotechnologische Forschung mbH (GBF)

(21) Appl. No.: 10/059,670

(22) Filed: Jan. 30, 2002

Related U.S. Application Data
(63) Continuation of application No. 09/441,239, filed on Nov. 16, 1999, now abandoned.
Application No. 09/441,239 is a continuation of application No. PCT/EP98/03056, filed on May 22, 1998.

(30) Foreign Application Priority Data
May 23, 1997 (EP) 97 108 403.3
Nov. 28, 1997 (EP) 97 120 941.6

(51) Int. Cl. A61K 39/00

(52) U.S. Cl. 424/185.1

(57) ABSTRACT

A common problem in human vaccinology is the limited availability of efficient and non-toxic adjuvants capable of promoting mucosal responses. The potential usefulness of fibronectin-binding protein (SfbI) of Streptococcus pyogenes as immunological adjuvant was assessed using ovalbumin (OVA) as a model antigen. Mice were immunized by intranasal route either with soluble OVA or OVA covalently coupled to SfbI. Immunization with OVA-SfbI resulted in the elicitation of about 100-fold higher titres of anti-OVA serum IgG than using OVA alone. The anti-OVA IgG subclass pattern was dominated in both groups of mice by IgG1, followed by IgG2b, IgG2a, and IgG3. Immunization with OVA-SfbI also resulted in the elicitation of OVA-specific IgA in lung washes (24% of the total IgA), which was absent in mice immunized with OVA alone. Spleen cells from OVA-SfbI immunized mice also gave a much stronger proliferative response to in vitro restimulation with soluble OVA. Phenotypic analysis of proliferating cells showed an enrichment in CD4+ T cells, producing a pattern of cytokines (IL-4, IL-5, IL-6 and IL-10) characteristic of Th2-type cells. In contrast to immunization with soluble OVA alone, OVA-SfbI induced the generation of CD8+ OVA-specific cytotoxic cells. These results demonstrate that SfbI represents a promising mucosal adjuvant able to substantially improve cellular, humoral and mucosal responses, when coupled to an antigen administered by intranasal route.

### CROSS-REFERENCE TO RELATE APPLICATION

This is a continuation of International Application No. PCT/EP98/03056 filed May 22, 1998, the entire disclosure of which is incorporated by reference.

### INTRODUCTION

Most pathogenic microorganisms are either restricted to the mucosal membranes or need to transit the mucosae during the early steps of infection (1). Therefore, it is desirable to obtain a local mucosal response as a result of vaccination in order to block both infection (i.e. colonization) and disease development (2). In addition, the development of systemic response is necessary to achieve protection against those pathogens that spread systemically. Parenterally administered vaccines are generally ineffective inducers of mucosal immunity, whereas mucosally delivered immunogens trigger both local and systemic immune responses (2, 3). Currently, the use of proteins or synthetic peptides in vaccine design bears the problem of their poor immunogenicity at the mucosal level. However, their administration together with a potent mucosal adjuvant would insure the elicitation of an efficient immune response.

Unfortunately, only a few molecules have been described which can exert activity as mucosal adjuvants (4-6), and their toxicity and potential side effects hinder their use in human vaccination (7, 8). Genetically inactivated derivatives of the heat labile toxin of Escherichia coli (LT) and the toxin produced by Vibrio cholera (CT) appear to be the most promising candidates (9, 10) although the domains of these toxins required for the biological activity are still controversial (11). However, it seems reasonable to hypothesize that optimal performance will require specific adjuvants depending on the individual antigen, the physio-pathogenesis of the infection and/or the characteristics of the host. Therefore, the availability of different adjuvants would allow to choose the best adjuvant for any particular situation. Thus, the identification of safe and efficacious adjuvants still constitutes a priority in the field of mucosal vaccines.

The attachment of Streptococcus pyogenes to epithelial cells is an essential event in the colonization and infection processes (12). The fibronectin binding protein I (SfbI) of S. pyogenes plays a key role in bacterial attachment, via fibronectin, to host cells and the subsequent colonization of the upper respiratory tract (13). More recently, it has been shown that SfbI also mediates internalization of S. pyogenes into non-phagocytic cells (14).

According to one embodiment, the problem underlying the invention is solved by a therapeutic or prophylactic composition comprising fibronectin-binding protein I (SfbI) as immunological adjuvant for any antigen(s) (immunogen(es)). As regards SfbI, reference is made to Infection & Immunity, 60 (1992) 3837-3844 and 65 (1997) 1357-1363.

The anchor domain and/or signal domain of SfbI can be missing or portions of SfbI can be used as far as these variants maintain their adjuvant effect.

The invention is based on the discovery that animals immunized by intranasal route developed strong humoral as well as mucosal antibody responses to SfbI as antigen.

According to the invention it can be shown that the presence of SfbI not only enhances humoral but also triggers mucosal antibody responses after intranasal immunization with soluble antigen such as ovalbumin (OVA) as a model antigen. In addition, OVA-specific CD8+ cytotoxic T lymphocytes (CTLs) are generated exclusively when SfbI is used as an adjuvant.

The composition according to the invention may comprise an antigen in addition to SfbI. Thus, the composition according to the invention may be characterized by a soluble antigen.

The composition according to the invention may be characterized in that the antigen and SfbI have been obtained by co-expression.

Further, the composition according to the invention may be characterized in that the antigen and the SfbI are coupled to each other, especially covalently coupled.

Further, the composition according to the invention may be characterized in that the antigen and SfbI form a fusion protein (quimeric protein), especially as expression product.

Further, the composition according to the invention may be characterized in that expression is carried out by means of a carrier strain, especially a vaccine carrier strain.

Further, the composition according to the invention may be characterized in that the antigen and SfbI have been expressed by different carrier strains.

The composition according to the invention may be designed for administration by mucosal and especially intranasal route.

### DISCUSSION

Since most of the pathogenic microorganisms have a mucosal portal of entry, an efficient immune response at the site of infection is required to prevent local infection, invasion and subsequent dissemination. The importance of developing strategies for the stimulation of the mucosal response is highlighted by the fact that secretory IgA is only produced by direct stimulation of the mucosal lymphoid compartment (27). The administration of soluble antigens as immunogens by this route has generally resulted in poor immune responses. This deficiency, however, can be overcome by the co-administration of adjuvants capable of enhancing the elicitated response. Unfortunately, only a few such adjuvants have been identified up to now. Furthermore, their intrinsic toxicity and side effects such as IgE production (7, 8) arise the problem of achieving a delicate equilibrium between inactivation and maintenance of their biological activity as mucosal adjuvant. Recent studies suggest that the newly developed attenuated derivatives of CT and LT (9, 10) may fulfill this requirement. Nevertheless, the identification of new mucosal adjuvants with different biological activities will allow to fine-tune the quality of the obtained response, thereby favouring the rational development of vaccines with optimal efficacy.

The results of this study demonstrate that administration of SfbI coupled to OVA by intranasal route enhanced the stimulation of antigen-specific humoral, cellular and mucosal immune responses. The overall effect of SfbI was approximately 100 fold enhancement of antigen-specific serum IgG response, the induction of antigen-specific secretory IgA, the priming of antigen-specific Th2-type CD4+ T cells and the generation of antigen-specific CTLs. Similar results were obtained when the activity of SfbI was evaluated using BALB/c mice, confirming that its adjuvancity is manifested regardless of the mouse H-2 haplotype.

Immunity against intracellular pathogens requires the generation of antigen-specific CTL able to recognize and destroy infected cells, thereby contributing to the clearance of the pathogenic microorganisms (23, 24). Parenteral immunization with CTL epitopes peptides has been reported to induce specific CTL responses (28, 29). However, little is known about the ability of mucosal immunization protocols for inducing antigen-specific CTL activity. Interestingly, intranasal immunization with OVA-SfbI, but not with soluble OVA, resulted in the generation of CD8+ OVA-specific CTLs. Therefore, SfbI may be used to deliver antigens by mucosal route when elicitation of a class I-restricted immune response is required.

There is an almost unanimous consensus that the adjuvancity of CT and LT at least requires an intact B and A2 subunits (30). In fact, recent studies of Hajishengallis et al. (31) demonstrate that appropriate folding is essential for optimal activity of quimeric proteins containing A2 domain since this domain should be inserted in the groove of the B subunit pentamer. The SfbI full-length polypeptide is constituted by 638 aa (32), however, the size of the biologically active adjuvant could be further reduced by elimination of the unnecessary domains (e.g. anchor and signal peptide). Therefore, SfbI exhibits a major potential as compared to CT or LT for the construction of chimeric proteins with vaccine antigens for mucosal immunization. In addition, the potential of SfbI is highlighted by the fact that in contrast to other adjuvants, SfbI is devoid of toxicity (i.e. inactivation is not required) and does not trigger an IgE response.

Several mechanisms may contribute to the adjuvancity of SfbI. A common characteristic to many mucosal adjuvants is their capacity to bind to receptors present on eucaryotic cells (33). In this regard, SfbI mediates the binding of S. pyogenes to epithelial cells via integrin-bound fibronectin (34). The binding of SfbI-antigen to the respiratory epithelium may increase the retention time of the immunogen, thereby facilitating antigen uptake and processing by resident antigen-presenting cells. Epitope presentation in association with class II molecules to resident CD4+ T cells may result in T cell activation and subsequent stimulation of B cells in the lung mucosa (35). This could explain the presence of OVA-specific IgA in lung washes from OVA-SfbI immunized mice, whereas the migration of higher number of OVA-primed CD4+ T cells to systemic lymphatic organs may explain the improved response of splenocytes after in vitro restimulation.

The underlying mechanism to the SfbI-mediated elicitation of CTLs remains to be elucidated. However, we have recently reported that SfbI is an invasin involved in the internalization of S. pyogenes within epithelial cells, and can mediate per se the uptake of inert latex particles (14). Therefore, we can hypothesize that soluble antigens coupled to SfbI may be taken up by antigen-presenting cells into a compartment with leakage into the cytoplasm (36-38). Consistent with this hypothesis, the loading of the cytoplasm with certain soluble antigens by pH-sensitive liposomes and osmotic strategies can induce antigen-specific CTLs, whereas immunizations with soluble proteins was ineffective (39-41).

It has been well documented that many bacterial components, which possess adjuvant activity can stimulate either macrophages, B or T lymphocytes (42, 43). Preliminary studies (data not shown) suggest that SfbI is a mitogen and a polyclonal activator of B cells in vitro. However, it seems unlikely that the mitogenic effect per se or any of the other postulated mechanisms may account for the complex activities of SfbI but rather a combination of them.

In conclusion, the presented data illustrate the potential of SfbI as a new tool for the development of efficient mucosal vaccines.

### EXAMPLE 1

Materials and Methods

Construction and purification of the SfbI His-Tag fusion protein: A 1581 bp BamHI/SalI fragment encompassing positions 274 to 1854 of the SfbI published DNA sequence (15) was cloned into pQE30 (Qiagen), thereby generating the recombinant plasmid pSTH2. This plasmid code for a His-Tag-SfbI fusion derivative (H2 fragment) lacking the N-terminal signal peptide and the C-terminal wall- and membrane-anchor regions. Expression and purification of His-Tag fusion protein from E. coli extracts under native conditions was performed according to Qiagen protocols. DNA manipulations were performed as described by Sambrook et al. (16), restriction and modification enzymes were supplied by New England Biolabs, Inc.

Conjugation procedure: The conjugation of OVA to SfbI was performed by using the heterobifunctional 3-(2-Pyridyldithio) propionic acid N-Hydroxysuccinimide ester (SPDP) as coupling reagent (17). In brief, OVA was dissolved in phosphate buffered saline (PBS) and activated with SPDP. The derivatising reaction was stopped with ethanolamine and the resulting product was extensively dialysed against PBS for removal of the cross-linker and the stop reagent. In the second step, the H2 fragment was added to the derivatized OVA and the resulting conjugate was concentrated on a DNA Speed Vac. DNA110 (Savant Instruments, Inc., Farmingdale, N.Y.). The efficacy of the coupling reaction was approximately 80% as determined by the spectrophotometric quantification (A₃₄₃) of the 2-thione released during the process (17).

Immunization: Pathogen-free, 7 weeks old C57BL/6 (H-2^{b}) female mice were obtained from Harlan Winkelmann. Group of five mice were immunized by an intranasal inoculation of 40 μl of PBS containing 1 mg/ml of OVA grade V (Sigma Chemical Co., St. Louis, Mo.) or 1 mg/ml of OVA coupled to a SfbI His-Tag fusion protein and received a booster 7 and 14 days after the primary immunization.

Sample collection: Serum samples were collected and monitored for OVA-specific IgM, IgG and IgA antibodies previous to immunization and at days 7, 14 and 28. At day 28 after immunization, mice were sacrificed and after per tracheal cannulation lung washes were collected, centrifuged at 3,000×g for 5 min to remove debris and stored at −20□ C. Spleen cells were isolated by gently forcing the tissue through a sterile stainless steel screen, erythrocytes were lysed with ammonium chloride (0.15 M NH₄Cl, 1.0 EM KHCO₃, 0.1 mM Na₂EDTA, pH 7.4), cell suspensions were washed and resuspended in RPMI 1640 supplemented with 10% of fetal calf serum (FCS), 100 U/ml penicillin, 50 μg/ml streptomycin, 5×10⁻⁵ M 2-mercaptoethanol and 1 mM L-glutamine (GIBCO BRL; Prisley, Scotland).

Antibodies assay: Antibody titres in serum and lung washes were determined by an enzyme-linked immunosorbent assay (ELISA). Nunc-Immuno MaxiSorp™ assay plates (Nunc, Roskilde, Denmark) were coated with 50 μl/well of OVA (5 μg/ml in 0.1 M Na₂HPO₄, pH 9.0) overnight at 4° C. After four washes (0.05% Tween 20 in PBS), they were blocked with 200 μl/well of 10% FCS in PBS for 1 h at 37° C. Serial two-fold dilutions of serum or lung washes were made in PBS containing 10% of FCS and 100 μl was added per well. The plates were incubated for 2 h at 37° C. After four washes, appropriate dilutions of either biotinylated μ-chain specific goat anti-mouse IgM, γ-chain specific goat anti-mouse IgG or α-chain specific goat anti-mouse IgA antibodies (Sigma, St. Louis, Mo.) or, to determine IgG subclass, biotin-conjugated rat anti-mouse IgG1, IgG2a, IgG2b and IgG3 (Pharmingen) were added and the plates were further incubated for 2 h at 37° C. After four washes, 100 μl of peroxidase-conjugated streptavidin (Pharmingen, St. Diego, Calif.) were added to each well and plates were incubated at room temperature (RT) for 1 h. After four washes reactions were developed at RT using ABTS [2,2′-Azino-bis-(3-ethybenzthiazoline-6-sulfonic acid)] in 0.1 M citrate-phosphate buffer (pH 4.35) containing 0.01% H₂O₂. Endpoint titers were expressed as the reciprocal log₂ of the last dilution which gave an optical density ∃0.1 above the values of the negative controls after a 30 min incubation. Total serum IgE was measured by the procedure described above using pairs of anti-mouse IgE (capture antibody: clone R35-72; detection antibody: clone R35-92) and a murine IgE standard (Pharmingen).

Cell proliferation assay: Spleen cells were adjusted to 2×10⁶ cells/ml in complete medium supplemented with 20 U/ml of human recombinant IL-2 (EUROCETUS; Chiron Co., Emeryville, USA), seeded at 100 μl/well in a flat-bottomed 96-well microtiter plate (Nunc, Roskilde, Denmark) and incubated for four days with three different concentrations of soluble OVA (10, 1, and 0.1 μg/ml). During the final 18 hours of culture 1 μCi of [³H]-thymidine (Amersham International, Amersham, U.K.) was added per well. The cells were harvested on paper filters (Filtermat A, WALLAC) using a cell harvester (Inotech, Wohlen, Switzerland) and the [³H]-thymidine incorporated into the DNA of proliferating cells was determined in a β-scintillation counter (WALLAC 1450, MICRO-β TRILUX).

FACScan analysis: Approximately 5×10⁵ cells were incubated in staining buffer (PBS supplemented with 2% FCS and 0.1% sodium azide) with the desired antibody or combination of antibodies for 30 min at 4° C. After washes, cells were analysed on a FACScan (Becton Dickinson). The monoclonal antibodies used were FITC-conjugated anti-CD4 (clone H129.19; Pharmingen), PE-conjugated anti-CD8ₐ (clone 53-6.7; Pharmingen) and FITC-conjugated goat anti-mouse IgG+IgM (Jackson Immunoresearch Laboratories, Inc., West Grove, Pa.)

Cytokines determination: Culture supernatants were collected from proliferating cells on day 4 and stored at −70° C. The determination of IL-2, IL-4, IL-5, IL-6, IL-10 and IFN-γ was performed by specific ELISA. In brief, 96-well microtiter plates were coated overnight at 4° C. with purified rat anti-mouse IL-2 mAb (clone JESG-1A12), anti-IL-4 mAb (clone 11B11), anti-IL-5 mAb (clone TRFK5), anti-IL-6 mAb (clone MP5-20F3), anti-IL-10 mAb (clone JES5-2A5), and anti-IFN-γ mAb (clone R4-6A2) (Pharmingen). After three washes, plates were blocked with 200 μl/well of PBS-10% FCS for 2 h at RT. Two-fold dilutions of supernatant fluids in PBS containing 10% FCS were added. A standard curve was generated for each cytokine using recombinant murine IL-2 (rIL-2), rIL-4, rIL-5, rIL-6, rIFN-γ, and rIL-10 (Pharmingen). Plates were further incubated at 4□ C. overnight. After washing, 100 μl/well of biotinylated rat against mouse IL-2 (clone JES6-5H4), IL-4 (clone BVD6-24G2), IL-5 (clone TRFK4), IL-6 (clone MP5-32C11), IL-10 (clone SXC-1) and INF-γ (clone XMG1.2) monoclonal antibodies were added and incubated for 45 min at RT. After six washes, streptavidin-peroxidase conjugated was added and incubated for 30 min at RT. Finally, the plates were developed using ABTS.

Cytotoxicity assay: Spleen cells were obtained from mice 14 days after the last immunization and 2×10⁶ effector cells were restimulated in vitro for 5 days in complete medium supplemented with 20 U/ml of rIL-2 and 10 μM of a peptide (pOVA; S I I N F E K L) encompassing a CTL determinant peptide from OVA (18). After restimulation, the assay was performed using the [³H]-thymidine incorporation method (19). In brief, 2×10⁶/ml of Ia⁻ EL4 thymoma cells derived from C57BL/6 mice were labelled with [³H]-thymidine for 4 h in either complete medium or complete medium supplemented with 20 μM of pOVA and used as target cells. Following washing, 2×10⁵ labelled targets and serial dilutions of effector cells were incubated in 200 μl of complete medium for 4 h incubation at 37° C. Cells were harvested and specific lysis was determined as follows:

[(retained c.p.m. in the absence of effectors)−(experimentally retained c.p.m. in the presence of effectors)/retained c.p.m. in the absence of effectors]×100.

Depletion of CD8+ spleen cells: CD8+ cell subset was depleted using MiniMACS Magnetic Ly-2 Microbeads according to the instructions of the manufacturer (Mitenyi Biotec GmbH, Germany). Depleted cell preparations contained # 1% CD8+ cells.

Statistical analysis: Results are reported as mean ∀ SEM. Statistical significance (p<0.05) between paired samples was determined by Student=s t test.

Results

Kinetics of serum OVA-specific antibody responses: Serum anti-OVA IgG and IgM antibody levels were assessed in mice immunized intranasally with soluble OVA or OVA-SfbI at different time intervals post-immunization. As shown in FIG. 1, only one dose of OVA or OVA-SfbI was sufficient to induce comparable OVA-specific IgM responses in serum after 7 days. The antigen-specific IgM titers remained relatively constant until termination of the experiment at day 28. On the other hand, OVA-specific IgG antibodies were initially detected at day 14, after two consecutive immunizations. The titer gradually increased throughout the period assessed, however, IgG responses were about 100 folds higher in the group immunized with OVA-SfbI. At day 28, IgG was the predominant OVA-specific immunoglobulin in the group immunized with OVA-SfbI, whereas the group immunized with soluble OVA exhibited comparable levels of antigen-specific IgG and IgM antibodies. Therefore, the presence of SfbI markedly enhanced serum IgG antibody responses to OVA. Neither OVA-specific serum IgA nor IgE antibodies were detected throughout the observation period in any of the groups.

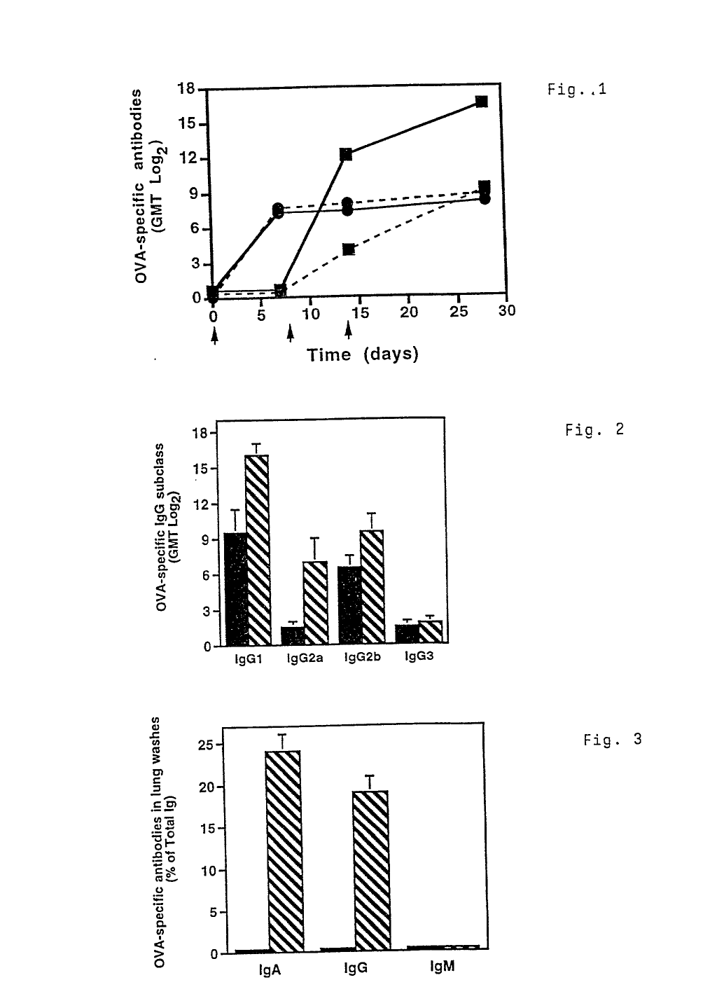

OVA-specific subclass profile of serum IgG: The subclass profile of OVA-specific serum IgG was determined two weeks after the last immunization (FIG. 2). Both groups of mice exhibited a similar IgG subclass profile with predominant IgG1 antibodies, followed by IgG2b, IgG2a and IgG3. However, the group immunized with soluble OVA showed significantly lower level of each isotype than the group of mice immunized with OVA-SfbI. This pattern of OVA specific IgG subclasses usually reflects the help provided by Th2-type CD4+ T cells (20).

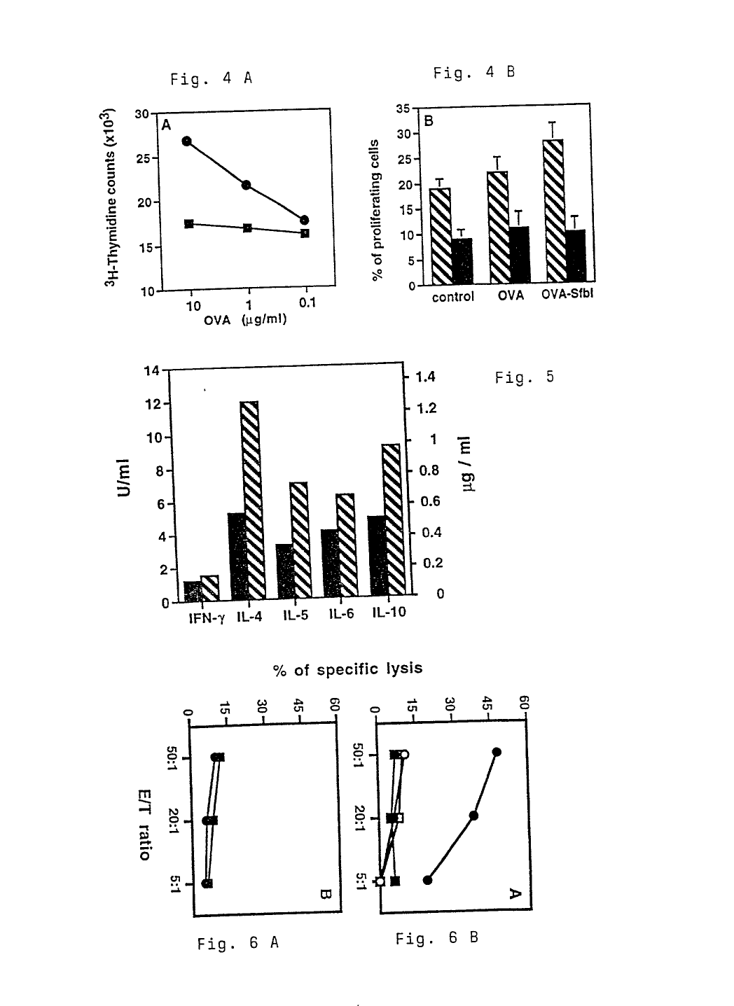

Effect of SfbI on the OVA-specific mucosal immune response in lungs after intranasal immunization: The stimulatory effect of SfbI on the mucosal immune response to OVA was assessed after three consecutive intranasal immunizations (FIG. 3). A significant increase in the anti-OVA IgA and IgG antibody titers was observed in lung washes after immunization with OVA-SfbI. In contrast, OVA-specific IgG and IgA were not detected after immunization with soluble OVA. Therefore, the presence of SfbI was essential to generate an antigen-specific local immune response in the lungs of immunized mice.

Characterization of the OVA-specific cellular immune response: To evaluate the ability of SfbI to promote an antigen-specific cellular immune response, spleen cells were isolated from mice intranasally immunized with either soluble OVA or OVA-SfbI. Splenocytes were cultured in the presence of soluble OVA during 4 days and specific proliferation was measured by [³H]-thymidine incorporation (FIG. 4A). The proliferative responses of antigen-stimulated spleen cells in vitro revealed marked differences between both groups. While cells from mice immunized with SfbI-OVA showed a strong proliferative response to OVA, antigenic restimulation at even the dose of 10 μg/ml failed to induce significant proliferation of spleen cells from mice immunized with soluble OVA. After 4 days of restimulation in vitro, FACScan analysis of proliferating cells revealed that cell cultures from OVA-SfbI immunized mice were more enriched in CD4+ T cells (FIG. 4B) when compared with cell cultures from non-immunized and OVA-immunized mice.

The presence of different cytokines in supernatant fluids from splenocytes cultured in vitro in the presence of soluble OVA (10 μg/ml) was also determined. The results showed that IL-4, IL-10, IL-5 and IL-6 were the predominant cytokines produced by splenocytes from mice immunized with OVA-SfbI. In contrast, IFN-γ was present in the supernatant fluid of cultured cells from both groups at a very low level and IL-2 was practically not detectable (date not shown). This characteristic Th2 cytokine pattern (21, 22) is consistent with the predominant OVA-specific IgG1 (20) found in serum of OVA-SfbI immunized mice. These findings further support the assumption that SfbI enhance antigen-specific antibody production by B cells by stimulating CD4+ Th2 cells.

SfbI-dependent OVA-specific class I-restricted CTL response: The CTLs constitute a potent effector mechanism that provide protection against many intracellular pathogens (23-25) and malignant cells (26). For the induction of cytotoxic CD8+ T cells, antigens must be expressed in target cells in association with MHC class I molecules. We analyzed whether intranasal administration of either soluble OVA or OVA-SfbI induced OVA-specific CTLs in immunized mice. Immunization with OVA-SfbI induced OVA specific CTLs response with 19, 38, and 48% of specific lysis of pOVA-pulsed target cells when tested at E:T ratios of 5:1, 20:1, and 50:1, respectively (FIG. 6A). CTL activity was peptide specific, as indicated by the lack of lysis of unloaded target cells. In contrast, immunization with soluble OVA did not trigger a specific CTL response (FIG. 6B).

To determine the nature of the OVA-specific CTL response observed after immunization with OVA-SfbI, effector cells were depleted of CD8+ T cells. The complete abrogation of antigen-specific cytotoxic activity observed after depletion of this cell population demonstrated that lysis was carried out by CD8+ T cells that recognize pOVA expressed on the surface of target cells.

### EXAMPLE 2

To assess the adjuvancity of SfbI when administered mixed with the vaccine antigen instead of covalently linked to it, mice were intranasally immunized with a solution of 40μl PBS containing 0.5 mg/ml of OVA or 0.5 mg/ml SfbI His-Tag fusion protein. Mice received a booster 7 and 14 days after the primary immunization.

Results

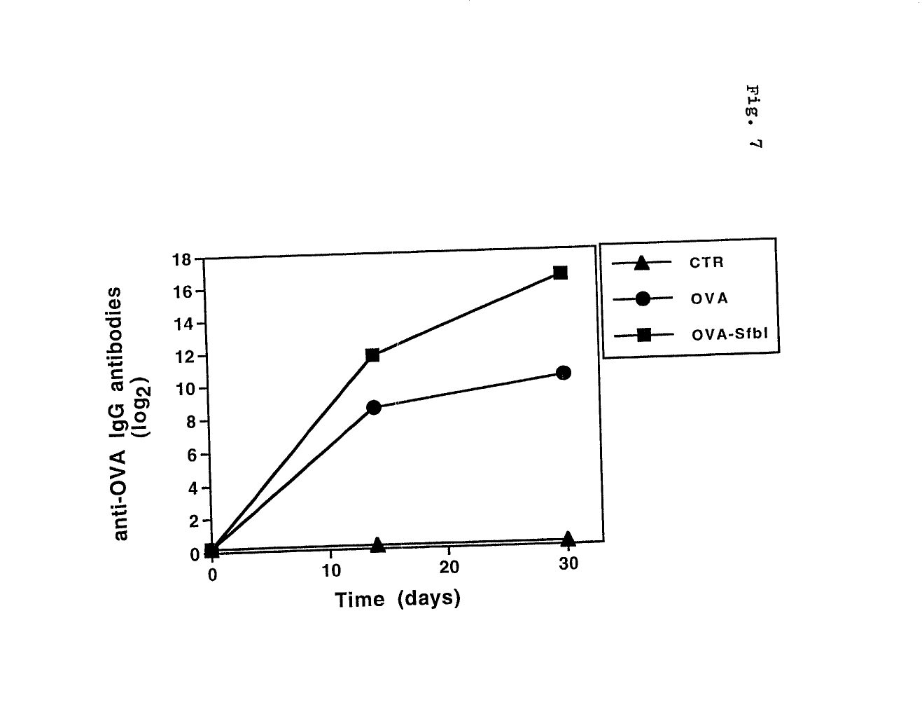

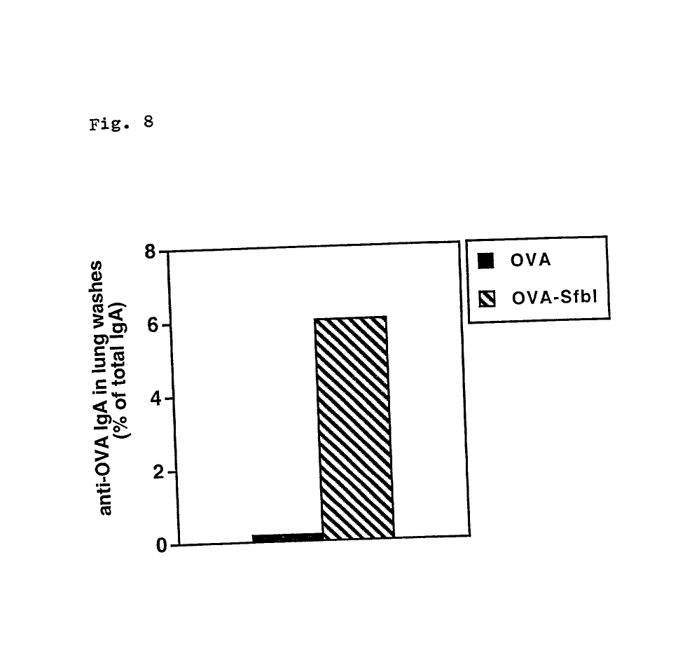

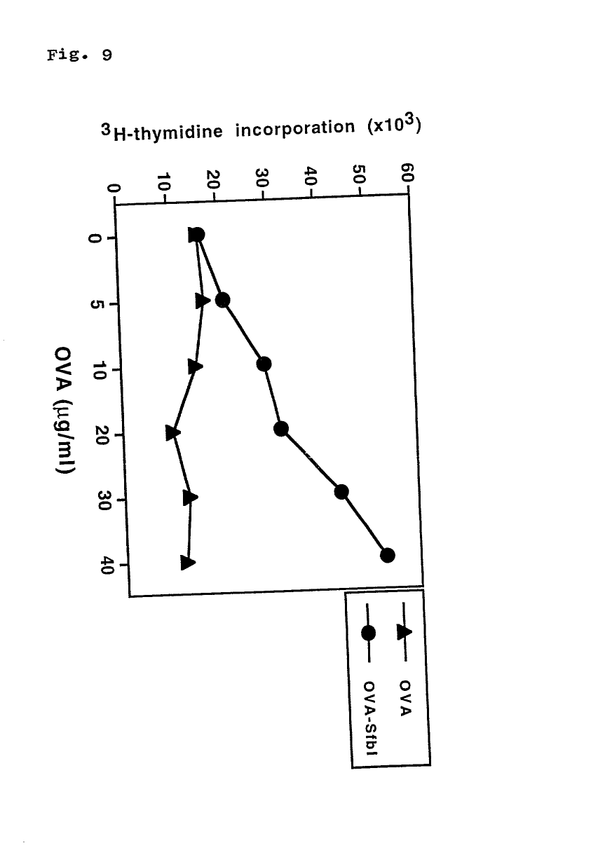

Humoral Responses: The obtained results show that the serum OVA specific IgG response was about 20-fold higher in the group immunized with OVA/SfbI than the group immunized with OVA alone (FIG. 7). OVA-specific IgA antibodies at the mucosal level were only detectable when SfbI was present in the vaccine mix (FIG. 8).

Cellular Responses: Splenocytes from mice immunized with OVA or with OVA/SfbI mixture were cultured in the presence of soluble OVA for 4 days and specific proliferation was measured by ³H-thymidine incorporation. While cells from mice immunized with OVA/SfbI showed strong proliferative response to OVA, non proliferation was observed in cultured cells from mice immunized with OVA alone (FIG. 9).

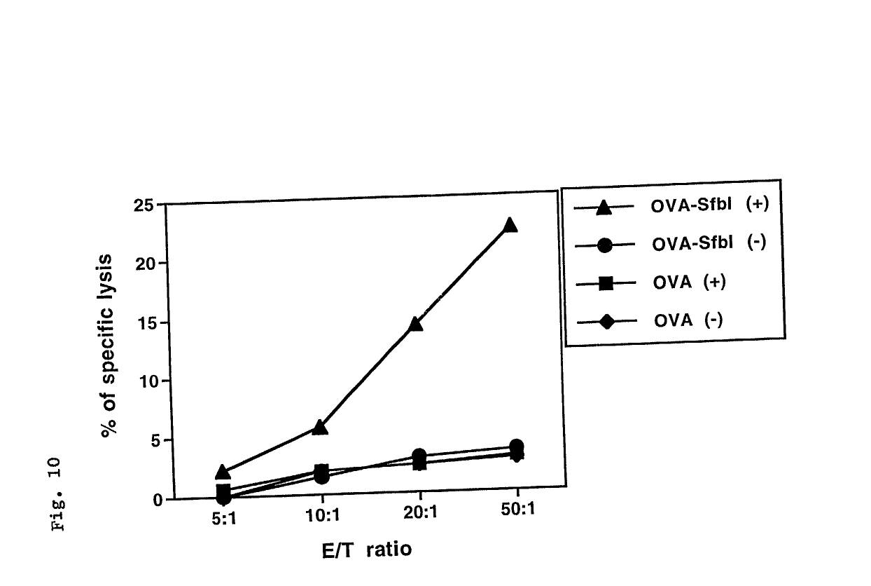

We analyzed whether administration of mixed OVA/SfbI was able to efficiently induce OVA-specific CTL response in immunized mice. The results show that intranasal immunization with OVA/SfbI mixed, but not with soluble OVA, resulted in the generation of OVA-specific CTL (FIG. 10).

Conclusions:

Co-administration of SfbI with vaccine antigens resulted in an enhancement of the humoral and mucosal antibody responses after intranasal immunization. In addition, the presence of SfbI in the vaccine preparation also facilitated the generation of antigen-specific CTL.

### REFERENCES

1. Levine, M. M., J. B. Kaper, R. E. Black, and M. L. Clements. 1983. New knowledge on pathogenesis of bacterial enteric infections as applied to vaccine development. Microbiol. Rev. 47:510.

2. Holmgren, J., C. Czerkinsky, N. Lycke, and A. M. Svennerholm. 1992. Mucosal immunity: implications for vaccine development. Immunobiology 184:157.

3. McGhee, J. R., J. Mestecky, M. T. Dertzbaugh, J. H. Eldridge, M. Hirasawa, and H. Kiyono. 1992. The mucosal immune system: from fundamental concepts to vaccine development. Vaccine 10:75.

4. Holmgren, J., N. Lycke and C. Czerkinsky. 1993. Cholera toxin and cholera B subunit as oral-mucosal adjuvant and antigen vector systems. Vaccine. 11:1179.

5. Roberts, M., A. Bacon, R. Rappuoli, M. Pizza, I. Cropley, G. Douce, G. Dougan, M. Marinaro, J. MacGhee, and S. Chatfield. 1995. A mutant pertussis toxin molecule that lacks ADP-ribosyltransferase activity, PT-9K/129G, is an effective mucosal adjuvant for intranasally delivered proteins. Infect. Immun. 63:2100.

6. Douce, G., C. Turcotte, I. Cropley, M. Roberts, M. Pizza, M. Domenghini, R. Rappuoli, and G. Dougan. 1995. Mutants of Escherichia coli heat-labile toxin lacking ADP-ribosyltransferase activity act as nontoxic, mucosal adjuvants. Proc. Natl. Acad. Sci. USA. 92:1644.

7. Snider, D. P., J. S. Marshall, M. H. Perdue, and H. Liang. 1994. Production of IgE antibody and allergic sensitization of intestinal and peripheral tissues after oral immunization with protein ag and cholera toxin. J. Immunol. 153:647.

8. Tamura, S., Y. Shoji, K. Hasiguchi, C. Aizawa, and T. Kurata. 1994. Effect of cholera toxin adjuvant on IgE antibody response to orally or nasally administered ovalbumin. Vaccine. 12:1238.

9. Yamamoto, S., Y. Takeda, M. Yamamoto, H. Kurazono, K. Imaoka, M. Yamamoto, K. Fujihashi, M. Noda, H. Kiyono, and J. R. MacGhee. 1997. Mutants in the ADP-ribosyltransferase cleft of cholera toxin lack diarrheagenicity but retain adjuvancity. J. Exp. Med. 185:1.

10. Yamamoto, S., H. Kiyono, M. Yamamoto, K. Imaoka, M. Yamamoto, K. Fujihashi, F. W. Van Ginkel, M. Noda, Y. Takeda, and J. R. MacGhee. 1997. A nontoxic mutant of cholera toxin elicits Th2-type responses for enhanced mucosal immunity. Proc. Natl. Acad. Sci. USA. In press.

11. Lycke, N., T. Tsuji, and J. Holmgren. 1992. The adjuvant effect of Vibrio cholera and Escherichia coli heat-labile enterotoxins is linked to their ADP-ribosyltransferase activity. Eur. J. Immunol. 22:2277.

12. Beachey, E. H. 1981. Bacterial adherence: adhesin-receptor interactions mediating the attachment of bacteria to mucosal surfaces. J. Infect. Dis. 143:325

13. Talay, S. R., P. Valentin-Weigand, P. G. Jerlstrom, K. N. Timmis, and G. S. Chhatwal. 1992. Fibronectin-binding protein of Streptococcus pyogenes: sequence of the binding domain involved in adherence of streptococci to epithelial cells. Infect. Immun. 60:3837.

14. Molinari, G., S. R. Talay, P. Valentin-Weigand, and G. S. Chhatwal. 1997. The fibronectin-binding protein of Streptococcus pyogenes, SfbI, is involved in the internalization of group A streptococci by epithelial cells. Infect. Immun. 65:1357.

15. Talay, S. R., P. Valentin Weigand, K. N. Timmis, and G. S. Chhatwal. 1994. Domain structure and conserved epitopes of SfbI protein, the fibronectin-binding adhesin of Streptococcus pyogenes. Mol. Microbiol. 13:531.

16. Sambrook, J., E. F. Fritsch, and T. Maniatis. 1989. Molecular cloning: a laboratory manual. 2nd ed.Cold Spring Harbor Laboratory, ed. Cold Spring Harbor Laboratory, Cold Spring Harbor, N.Y.

17. Carlsson, J., H. Drevin, and R. Axen. 1983. Protein Thiolation and reversible protein-protein conjugation. N-succinimidyl-3-(2-pyridyl-dithio) propionate, a new heterobifunctional reagent. Biochem. J. 173:723.

18. Rotzschke, O., K. Falk, S. Stevanovic, G. Jung, P. Walden, and H. G. Ranmensee. 1991. Exact prediction of a natural T cell epitope. Eur. J. Immunol. 21:2891.

19. Matzinger, P. 1991. The JAM test. A simple assay for DNA fragmentation and cell death. J. Immunol. Methods 145:185.

20. Stevens, T. L., A. Bossie, V. M. Sanders, R. Fernandez Botran, R. L. Coffman, T. R. Mosmann, and E. S. Vitetta. 1988. Regulation of antibody isotype secretion by subsets of antigen-specific helper T cells. Nature 334:255.

21. Mosmann, T. R. and R. L. Coffman. 1989. TH1 and TH2 cells: different patterns of lymphokine secretion lead to different functional properties. Annu. Rev. Immunol. 7:145.

22. Street, N. E. and T. R. Mosmann. 1991. Functional diversity of T lymphocytes due to secretion of different cytokine patterns. FASEB J. 5:171.

23. Whitton, J. L., P. J. Southern, and M. B. Oldstone. 1988. Analyses of the cytotoxic T lymphocyte responses to glycoprotein and nucleoprotein components of lymphocytic choriomeningitis virus. Virology 162:321.

24. Kast, W. M., R. Offringa, P. J. Peters, A. C. Voordouw, R. H. Meloen, A. J. van der Eb, and C. J. Melief. 1989. Eradication of adenovirus E1-induced tumours by E1A-specific cytotoxic T lymphocytes. Cell 59:603.

25. Walker, B. D., C. Flexner, K. Birch Limberger, L. Fisher, T. J. Paradis, A. Aldovini, R. Young, B. Moss, and R. T. Schooley. 1989. Long-term culture and fine specificity of human cytotoxic T-lymphocyte clones reactive with human immunodeficiency virus type. Proc. Nati. Acad. Sci. U. S. A. 86:9514.

26. Lynch, D. H., and R. E. Miller. 1991. Immunotherapeutic elimination of syngeneic tumours in vivo by cytotoxic T lymphocytes generated in vitro from lymphocytes from the draining lymph nodes of tumour-bearing mice. Eur. J. Immunol. 21:1403.

27. Underdown, B. J. and J. M. Schiff. 1986. Immunoglobulin A: strategic defence initiative at the mucosal surface. Annu. Rev. Immunol. 4:389.

28. Nehete, P. N., K. S. Casement, R. B. Arlinghaus, and K. H. Sastry. 1995. Studies of in vivo induction of HIV-1 envelop-specific cytotoxic T lymphocytes by synthetic peptides from the V3 loop region of HIV-1 IIIB gp120. Cell. Immunol. 160:217.

29. Mandelboim, O., G. Berke, M. Fridkin, M. Feldman, M. Einsenstein, and L. Einsebach. 1994. CTL induction by a tumour associated antigen octapeptide derived from a murine lung carcinoma. Nature. 369:67.

30. Lycke, N., T. Tsuji, and J. Holmgren. 1992. The adjuvant effect of Vibrio cholera and Escherichia coli heat-labile enterotoxin is linked to their ADP-ribosyltransferase activity. Eur. J. Immunol.22:2277.

31. Hajishengallis, G., E. Harokopakis, T. E. Greenway, and S. M. Michalek. 1997. Intranasal immunization with recombinant Gram-Negative bacterial vectors. 97th General Meeting American Society for Microbiology. 256:91-E. (Abstract).

32. Talay, S. R., E. Ehrenfeld, G. S. Chhatwal, and K. N. Timmis. 1991. Expression of the fibronectin-binding components of Streptococcus pyogenes in Escherichia coli demonstrates that they are proteins. Mol. Microbiol. 5:1727.

33. De Aizpurua, H. J. and G. J. Russell Jones. 1988. Oral vaccination. Identification of classes of proteins that provoke an immune response upon oral feeding. J. Exp. Med. 167:440.

34. Courtney, H. S., I. Ofek, W. A. Simpson, D. L. Hasty, and E. H. Beachey. 1986. Binding of Streptococcus pyogenes to soluble and insoluble fibronectin. Infect. Immun. 53:454.

35. Coffman, R. L., B. W. Seymour, D. A. Lebman, D. D. Hiraki, J. A. Christiansen, B. Shrader, H. M. Cherwinski, H. F. Savelkoul, F. D. Finkelman, M. W. Bond, and et al. 1988. The role of helper T cell products in mouse B cell differentiation and isotype regulation. Immunol. Rev. 102:5.

36. Norbury, C. C., L. J. Hewlett, A. R. Prescott, N. Shastri, and C. Watts. 1995. Class I MHC presentation of exogenous soluble antigen via macropinocytosis in bone marrow macrophages. Immunity. 3:783.

37. Verma, N. K., H. K. Ziegler, M. Wilson, M. Khan, S. Safley, B. A. D. Stocker, and G. K. Schoolnik. 1995. Delivery of class I and class II MHC-restricted T-cell epitopes of listeriolysin of Listeria monocytogenes by attenuated Salmonella. Vaccine. 13:142.

38. Pfeifer, J. D., M. J. Wick, R. L. Roberts, K. Findlay, S. J. Normark, and C. V. Harding. 1993. Phagocytic processing of bacterial antigens for class I MHC presentation to T cells. Nature. 361:359.

39. Zhou, F., S. C. Watkins, and L. Huang. 1994. Characterization and kinetics of MHC class I-restricted presentation of a soluble antigen delivered by liposomes. Immunobiology. 190:35.

40. Moore, M. W., F. R. Carbone, and M. J. Bevan. 1988. Introduction of soluble protein into the class I pathway of antigen processing and presentation. Cell 54:777.

41. Carbone, F. R. and M. J. Bevan. 1990. Class I-restricted processing and presentation of exogenous cell-associated antigen in vivo. J. Exp. Med. 171:377.

42. Alouf, J. E., H. Knoll, and W. Kahler. 1991. The family of mitogenic, shock-inducing and superantigenic toxins from staphylococci and streptococci. In Sourcebook of bacterial protein toxins. J. E. Alouf and J. H. Freer, eds. Academic Press, Inc. San Diego, Calif., p. 367.

43. Andersson, J., S. Nagy, L. Bjork, J. Abrams, S. Holm, and U. Andersson. 1992. Bacterial toxin-induced cytokine production studied at the single-cell level. Immunol. Rev. 127:69.

Legends

FIG. 1. Kinetics of OVA-specific IgG ( ) and IgM ( ) antibodies in serum of mice (n=5) immunized by the intranasal route with either OVA (—) or OVA-SfbI (—). Antibody titers were determined in serum at day 0, 7, 14 and 28. Results are expressed as the reciprocal Log₂ of the geometric mean end point titer (GMT), SEM was in all cases lower than 10%. Immunizations are indicated by arrows.

FIG. 2. Subclass profile of the OVA-specific IgG antibodies present in the serum of mice (n=5) intranasally immunized with OVA (solid bars) or OVA-SfbI (hatched bars) at day 28 postimmunization. Immunizations with either OVA or OVA-SfbI resulted predominantly in IgG1 antibodies, followed by IgG2b, IgG2a and IgG3. Results are expressed as GMT ∀ SEM.

FIG. 3. OVA-specific antibody response in lung washes after mucosal immunization. Effect of SfbI on the local immune response in the lungs of mice after three consecutive immunizations with OVA (solid bars) or OVA-SfbI (hatched bars). The total and OVA-specific IgA, IgG, and IgM antibodies were measured in the lung lavages of both groups of mice (n=5) at day 28 after the first immunization. Results are expressed as percentage of OVA-specific antibodies with respect to the total immunoglobulin isotype. SEM are indicated by vertical lines.

FIG. 4. (A) Proliferative response of antigen-specific spleen cells from mice immunized with OVA ( ) or with OVA-SfbI ( ) to in vitro restimulation with soluble OVA (A). The values are expressed as mean c.p.m. ∀ SEM of triplicates. Background values were obtained from wells without the stimulating antigen. (B) Flow cytometric analysis of OVA-specific proliferating CD4+ (hatched bars) and CD8+ (solid bars) T cells. SEM are indicated by vertical lines.

FIG. 5. Profile of cytokines in supernatant fluids from cultured spleen cells from mice immunized with OVA (solid bars) or OVA-SfbI (hatched bars). Cytokine production was determined by ELISA. Results represent the means of three determinations (U/ml for IFN-γ, IL-4, IL-5, and IL6; and μg/ml for IL-10) ∀ SEM.

FIG. 6. Recognition of MHC-I-restricted pOVA epitope by lymphocytes primed in vivo in mice by intranasally administered OVA-SfbI (A) or soluble OVA (B). Spleen cells from immunized mice were restimulated in vitro 5 days in the presence of pOVA (20 μM). At the end of the culture lymphocytes were tested in a [³H]-thymidine-release assay using EL-4 ( ) and pOVA-loaded EL-4 ( ) as targets. Effect of CD8+ T cell depletion in the lysis of EL-4 (φ) or EL-4+pOVA (Γ) by effector cells from OVA-SfbI immunized mice. Results are mean values of triplicates and are expressed as: [(retained c.p.m. in the absence of effectors)−(experimentally retained c.p.m. in the presence of effectors)/retained c.p.m. in the absence of effectors]×100; SEM were lower than 5% of the values.

1. Therapeutic or prophylactic composition comprising fibronectin-binding protein I (SfbI) as an immunological adjuvant for any antigen or immunogen.

2. Composition of claim 1 wherein said SfbI lacks at least one of its anchor domain and signal domain.

3. Composition of claim 1 comprising at least one portion of SfbI.

4. Composition of claim 1 comprising an antigen in addition to SfbI.

5. Composition of claim 4 comprising a soluble antigen.

6. Composition of claim 4 wherein the antigen and SfbI have been obtained by co-expression.

7. Composition of claim 6 wherein expression is carried out by means of a carrier strain.

8. Composition of claim 7 wherein expression is carried out by news of a vaccine carrier strain.

9. Composition of claim 4 wherein the antigen and the SfbI are coupled to each other.

10. Composition of claim 4 wherein the antigen and SfbI form a fusion protein (chimeric protein).

11. Composition of claim 10 wherein the antigen and SfbI form an expression product.

12. Composition of claim 10 wherein expression is carried out by means of a carrier strain.

13. Composition of claim 12 wherein expression is carried out by means of a vaccine carrier strain.

14. Composition of claim 1 wherein the antigen and SfbI have been expressed by different carrier strains.

15. Composition of claim 2 wherein the antigen and SfbI have been expressed by different carrier strains.

16. Composition of claim 3 wherein the antigen and SfbI have been expressed by different carrier strains.

17. Composition of claim 4 wherein the antigen and SfbI have been expressed by different carrier strains.

18. Composition of claim 5 wherein the antigen and SfbI have been expressed by different carrier strains.

19. Composition of claim 6 wherein the antigen and SfbI have been expressed by different carrier strains.

20. Composition of claim 7 wherein the antigen and SfbI have been expressed by different carrier strains.

21. Composition of claim 1 adapted for a administration by a mucosal route.

22. Composition of claim 15 adapted for administration by an intranasal route.
